(19) United States
(12) Patent Application Publication
(10) Pub. No.: US 2017/0354533 A1
(43) Pub. Date: Dec. 14, 2017

(54) HEATED AIR ECTOPARASITE TREATMENT DEVICES AND RELATED METHODS

(71) Applicant: Larada Sciences, Inc., Salt Lake City, UT (US)

(72) Inventor: Kelly J. Brodbeck, Santa Barbara, CA (US)

(21) Appl. No.: 15/177,054

(22) Filed: Jun. 8, 2016

(51) Int. Cl. A61F 7/00; A45D 20/12

(52) U.S. Cl. CPC A61F 7/0085; A45D 20/122; A61F 2007/0086; A61F 2007/0087; A61F 2007/0008; A61F 2007/0096; A61F 2007/0052; A61F 2007/006

(57) ABSTRACT

Ectoparasite treatment devices utilizing heated air and related treatment methods. In some embodiments, the device may comprise a heating assembly positioned within a housing that is configured to generate heated air and a blower, such as a fan, for moving the heated air. The device may further comprise an air diffuser configured to receive heated air from the blower and to mix the heated air to increase uniformity of temperature of the heated air. A tip comprising a plurality of tines with ports may be removably coupled with the housing. The tip may be fluidly coupled with the diffuser. A temperature sensor and temperature control electronics may be configured to use temperature data and adjust the heating assembly such that the heated air delivered through the tip is maintained between a minimum temperature of about 54 degrees Celsius and a maximum temperature of about 62 degrees Celsius.

### SUMMARY

Disclosed are embodiments of heated air treatment devices used for delivering heated air to the scalp and/or hair of humans and/or animals to be used for treatment of ectoparasite infestations, along with various implementations of methods for treatment of such infestations. In preferred embodiments, the device comprises a handheld device configured to enable an operator (such as a parent or health-care provider) to hold the device in one hand during operation and use the other hand to manipulate the subject's hair to facilitate the treatment. Various features, elements, components, and/or steps are disclosed in connection with the preferred embodiments and implementations discussed below. However, it is anticipated that numerous alternative features, elements, components, and/or steps, or alternative combinations of such features, elements, components, and/or steps, will be apparent to those of ordinary skill in the art after having received the benefit of this disclosure.

In an example of preferred embodiment of an ectoparasite treatment device, the device may comprise a handle configured to allow an operator to grip the handle and operate the ectoparasite treatment device. In some embodiments, the ectoparasite treatment device may comprise a handheld device and may therefore comprise a handle configured to allow an operator to grip the handle and operate the ectoparasite treatment device with a single hand. A housing may be coupled with the handle and a heating assembly may be positioned within the housing. The heating assembly may be configured to generate heated air for treatment of ectoparasites.

The device may further comprise a blower and an air diffuser positioned within the housing and configured to receive heated air from the blower. The air diffuser may be configured to mix the heated air to increase uniformity of temperature of the heated air.

A tip may be coupled with the housing. In some embodiments, the tip may be removable and/or disposable from the housing, or otherwise from the device, such that new tips may be used for each treatment, or after a desired/allotted treatment time with a particular tip. The tip may comprise a plurality of tines. One or more (in some embodiments, all) of the tines may comprise ports configured to deliver heated air therethrough. The tip may be fluidly coupled with the air diffuser and may be configured to be removable from the ectoparasite treatment device.

The device may further comprise one or more temperature sensors configured to sense a temperature of the heated air and may further comprise temperature control electronics, such as a controller and/or PCB, which may be operatively coupled with the temperature sensor and the heating assembly. The temperature control electronics may be configured to receive temperature data from the temperature sensor and adjust the heating assembly such that the heated air delivered through the tip is maintained between a minimum temperature of about 54 degrees Celsius and a maximum temperature of about 62 degrees Celsius, as measured at each of the ports. In some embodiments, the temperature control electronics may be configured to receive temperature data from the temperature sensor and adjust the heating assembly such that the heated air delivered through the tip is maintained between a minimum temperature of about 56 degrees Celsius and a maximum temperature of about 59 degrees Celsius, as measured at each of the ports.

In some embodiments, the air diffuser may comprise a first set of apertures configured to receive heated air therethough, a mixing chamber fluidly coupled with the first set of apertures, and a second set of apertures configured to deliver heated air from the mixing chamber out of the air diffuser, wherein the first set of apertures is misaligned with respect to the second set of apertures.

Some embodiments may further comprise a memory element, which in some embodiments may be part of a microchip, positioned on the tip, wherein the memory element is configured to receive and store tip usage parameter data. Some embodiments may further comprise a controller configured to be communicatively coupled with the memory element when the tip is coupled with the housing. The controller may be configured to receive tip usage parameter data and alter at least one function of the ectoparasite treatment device in accordance with the tip usage parameter data. In some embodiments, the temperature control electronics may also serve as the controller.

The tip usage parameter data may comprise, for example, tip usage time. In some such embodiments, the controller may be configured to receive the tip usage time from the memory element and, upon determining that the tip usage time has exceeded a threshold treatment time, alter the at least one function of the ectoparasite treatment device.

In some embodiments, the controller may be configured to alter the at least one function of the handheld ectoparasite treatment device by at least one of storing an encrypted code on the memory element indicating that the tip usage time has expired; disabling the ectoparasite treatment device for use with the tip; generating an audible notification; generating a visible notification; and transmitting a wireless signal indicating that the threshold treatment time has been exceeded.

In another example of an ectoparasite treatment device according to other embodiments, the device may comprise a heating assembly configured to generate heated air for treatment of ectoparasites; a blower; and an air diffuser configured to receive heated air from the blower and to mix the heated air to increase uniformity of temperature of the heated air. The device may further comprise a tip comprising a plurality of elongated tines, wherein at least a subset of the tines comprises ports configured to deliver heated air therethrough. In some embodiments, a first subset of the ports are each configured to direct heated air in a first direction relative to a cross-section of the tip extending perpendicular to an axis of the tines, and a second subset of the ports are each configured to direct heated air in a second direction extending at a first angle relative to the first direction. The tip may further comprise a third subset of the ports that are each configured to direct heated air in a third direction extending at a second angle relative to the first direction and in an opposite direction relative to the first angle, and wherein the tip is fluidly coupled with the air diffuser. In some embodiments, each of the ports is configured such that at least substantially no heated air is directed in a direction opposite from the first direction.

Some embodiments may further comprise one or more temperature sensors configured to sense a temperature of the heated air. Temperature control electronics may be operatively coupled with the temperature sensor(s) and the heating assembly. The temperature control electronics may be configured to receive temperature data from the temperature sensor(s) and adjust the heating assembly such that the heated air delivered through the tip is maintained between a minimum temperature of about 54 degrees Celsius and a maximum temperature of about 62 degrees Celsius, as measured at each of the ports or, more preferably between about 56 degrees Celsius and about 59 degrees Celsius.

In some embodiments, the tip may be configured to be removable from the handheld ectoparasite treatment device and/or may be disposable such that the ectoparasite treatment device is configured to allow for being coupled with a different tip following use of the first tip during treatment.

In some embodiments, the air diffuser may comprise a first set of apertures configured to receive heated air therethough; a mixing chamber fluidly coupled with the first set of apertures; and a second set of apertures configured to deliver heated air from the mixing chamber out of the air diffuser. The first set of apertures may be misaligned with respect to the second set of apertures and/or may comprise apertures having at least one of a different shape and a different size relative to the second set of apertures.

In an example of a method for treating an ectoparasite infestation according to some implementations, the method may comprise obtaining an ectoparasite treatment device configured to deliver heated air to desiccate ectoparasites and ectoparasite eggs. In some implementations, the ectoparasite treatment device may comprise a housing; and a tip comprising a plurality of tines, wherein at least a subset of the tines comprises ports configured to deliver heated air therethrough. The method may further comprise coupling the tip with the housing, or another part of the device, and validating a tip usage parameter associated with the tip upon coupling the tip with the housing/device. The method may further comprise using the ectoparasite treatment device to apply heated air to the hair and/or scalp of a subject, such as a human patient, during a treatment session. Preferably the heated air is maintained between a minimum temperature of about 54 degrees Celsius and a maximum temperature of about 62 degrees Celsius during the treatment session. Even more preferably, the heated air is maintained between a minimum temperature of about 56 degrees Celsius and a maximum temperature of about 59 degrees Celsius during the treatment session.

In some implementations, the tip usage parameter may comprise a treatment time.

In some implementations, the step of validating the tip usage parameter may comprise transmitting the treatment time from a memory element on the tip; comparing the treatment time with a threshold treatment time; and, upon determining that the treatment time has not exceeded the threshold treatment time, allowing the ectoparasite treatment device to operate.

Some implementations may further comprise, upon determining that the treatment time has exceeded the threshold treatment time, performing one or more of storing an encrypted code on the memory element indicating that usage time for the tip has expired; disabling the ectoparasite treatment device for use with the tip; generating an audible notification; generating a visible notification; and transmitting a wireless signal indicating that the threshold treatment time has been exceeded.

In some implementations, a usage time of the ectoparasite treatment device may be tracked during the treatment session and may be stored on a component of the tip, such as on a microchip and/or memory element on the chip. This data may be stored during the treatment session or, alternatively, may be stored following termination of a treatment session, for example.

Some implementations may further comprise removing the tip from the housing; coupling a tip with a second ectoparasite treatment device; and transmitting the usage time from a memory element on the tip to a controller or other suitable component of the second ectoparasite treatment device. In some such implementations, the method may further comprise comparing the usage time with a threshold treatment time; and upon determining that the usage time has not exceeded the threshold treatment time, allowing the second ectoparasite treatment device to operate.

The features, structures, steps, or characteristics disclosed herein in connection with one embodiment may be combined in any suitable manner in one or more alternative embodiments.

### BRIEF DESCRIPTION OF THE DRAWINGS

Understanding that drawings depict only certain preferred embodiments and are not therefore to be considered to be limiting in nature, the preferred embodiments will be described and explained with additional specificity and detail through the use of the accompanying drawings in which:

FIG. 1 is a perspective view of a heated air ectoparasite treatment device according to some embodiments;

FIG. 2 is a cross-sectional view taken along line 2-2 in FIG. 1;

FIG. 3 is an exploded view of the heated air ectoparasite treatment device of FIG. 1;

FIG. 4 is a close-up, perspective view of a removable tip configured to be coupled with a heated air ectoparasite treatment device according to some embodiments;

FIG. 5 is a top plan view of the removable tip of FIG. 4 with arrows schematically depicting how heated air is configured to be delivered through the ports of the tip;

FIG. 6 is a perspective view of a diffuser according to some embodiments for mixing heated air to improve the uniformity of the air temperature shown from a proximal side of the diffuser;

FIG. 7 is a perspective view of the diffuser shown in FIG. 6 but shown from the distal side of the diffuser; and

FIG. 8 is a flow chart depicting a method for treating an ectoparasite infestation according to some implementations.

### DETAILED DESCRIPTION

In the following description, numerous specific details are provided for a thorough understanding of specific preferred embodiments. However, those skilled in the art will recognize that the invention can be practiced without one or more of the specific details, or with other methods, components, structures, etc.

In some cases, well-known structures, details, or operations are not shown or described in detail in order to avoid obscuring aspects of the preferred embodiments. Furthermore, the described features, structures, or characteristics may be combined in any suitable manner in one or more embodiments.

Disclosed are embodiments of heated air treatment devices used for delivering heated air to the scalp and/or hair of humans and/or animals to be used for eliminating ectoparasites, such as lice and lice eggs, for example, from such a patient or animal. In preferred embodiments, the device comprises a handheld device configured to enable an operator (such as a parent or health-care provider) to hold the device in one hand during operation and use the other hand to manipulate the subject's hair to facilitate the treatment.

Unlike typical prior devices, some preferred embodiments may further lack any tubing for delivery of the heated air from a heating element to a port. Instead, the air may be heated entirely within a relatively small housing that, as mentioned above, may be configured to allow for holding by a single hand during treatment. Some embodiments may be configured to provide a very precise range of temperatures of the heated air, so that the air may effectively eliminate the ectoparasites and associated eggs by desiccation but without burning the subject's scalp/skin. Thus, some preferred embodiments may be configured to maintain the heated air between about 54 degrees and about 62 degrees Celsius, as measured at one or more of the exit ports for the heated air formed on the device, such as on ports formed within one or more tines of a tip of the device. In still more preferred embodiments, the device may be configured to maintain the heated air between about 56 degrees and about 59 degrees Celsius, as measured at one or more of the exit ports for the heated air formed on the device. In some embodiments, the device may be configured to control the temperature delivered at all ports within one or both of the ranges mentioned above (again, as measured right at the exit port).

In some embodiments, the device may be light in weight. More particularly, the device may preferably be at or less than about two pounds in weight, and even preferably at or less than about one pound in weight. In addition, overheating discomfort may be mitigated by a technique of slowly moving the applicator tip in a short radius circle (preferably about ½ inch) to take the sting out of the treatment. The ability to hold the tip apparatus against the scalp without too much force on the scalp, and slowly rotate it, requires a certain amount of operator finesse and device control. This is why preferred embodiments of the design are well-balanced, easy to hold, lightweight, and not awkwardly long.

In some embodiments, a chamber may be provided within a housing of the device within which the air may be heated and/or mixed. A heating element, a diffuser element, and/or secondary motorized fan or stator diffuser may be positioned within the mixing chamber. In this manner, the air may be heated and sufficiently mixed to allow for precise control over the temperature of the air exiting the device, as previously mentioned. Preferably, the mixing chamber or an equivalent mixing zone comprises a heating element, a diffuser element, and a fan element. By providing a suitable combination of such elements, the length/size of the mixing chamber/zone may be minimized while still allowing for precise temperature control.

In some embodiments, the heated air delivered through the tip of the device may be between about 1 CFM and about 50 CFM. In some preferred embodiments, the heated air delivered through the tip may be between about 10 CFM and about 30 CFM.

To further facilitate the desired, precise temperature control, some embodiments may incorporate temperature control loop functionality. Such functionality may be implemented using hardware, software, firmware, and/or any combination thereof. Such elements may be employed to constantly adjust the power to the heating element and/or fan. Thus, preferably one or more temperature sensors are provided within the device, and even more preferably within the mixing chamber/zone of the device. By sensing temperatures frequently, the software can make adjustments frequently. Similarly, if the heating element and/or fan adjustments are small, precise temperature control may be provided. In some preferred embodiments, the temperature sensor(s) are configured with an accuracy of no more than about +/−0.1° C. Similarly, in preferred embodiments and implementations, temperature adjustment frequency may be at least once every 1 second. More preferably, temperature adjustment frequency may be at least once every 0.1 seconds. Even more preferably, temperature adjustment frequency may be at least once every 1/60th of a second (60 Hz).

Some embodiments may incorporate a proportional-integral-derivative (“PID”) algorithm. For example, some embodiments may comprise a PID controller or another control loop feedback mechanism. In combination with one or more of the other features and/or elements described herein, precise temperature control may be provided, which has been difficult to achieve in small devices.

Several factors may contribute to the ability of various preferred embodiments of the device to provide precision temperature control. For example, preferably the heated air is mixed thoroughly before it gets to the scalp. Thus, in preferred embodiments, the heating chamber, the tube that leads to the tip, and/or the tip are configured to facilitate mixing of a hot and cold air to a high degree of uniformity.

In addition, preferably the temperature sensor(s) should be carefully positioned. For example, the temperature sensor(s) ideally should not be located right at the scalp where it could be damaged. It should instead be positioned back from the scalp where the air might not be fully mixed. It may therefore be important to find a location where the air temperature at the sensor is the same as the air temperature at the scalp while protecting the sensor from damage.

The temperature sensor should also be highly accurate. Because there are many potential error sources, such as uneven mixing of the air, poor location of the temperature sensor(s), sensor inaccuracies, electronics inaccuracies, and inaccuracies of the software algorithm (PID, e.g.) that control the temperature, it is important that one or more (in some cases, all) of these parameters are controlled to improve the ability of the device to maintain a very tight and accurate temperature range.

Use of small/handheld devices, may introduce additional difficulties. For example, it has been discovered that optimizing spot treatment times can be done by eliminating, or at least reducing, the overlap used in the applicator tip during treatment. This may allow for reduction of treatment times per spot. For example, in some preferred implementations, the treatment time per spot may be less than about 30 seconds. In some such implementations, the treatment time per treatment spot may be less than about 25 seconds, and even more preferably less than about 20 seconds. However, use of a smaller applicator tip, as in certain preferred embodiments disclosed herein, in combination with treatment techniques designed to minimize treatment spot overlap, may run the risk of missing an area of the scalp where lice or other ectoparasites, or their associated eggs, are located. If the eggs are not appropriately dehydrated, they will hatch and the infestation may return.

In order to overcome or reduce such problems, some embodiments may incorporate a specifically designed tip comprising ports directing heated air in particular directions. For example, some embodiments may comprise a tip having a plurality of tines. One or more (in some cases, all) of the tines may comprise ports configured to deliver at least some of the heated air toward a treatment side of the tip (taken from a cross-sectional view of a plane extending through each of the tines of the tip) and at least some of the heated air away from the treatment side, such as to adjacent areas relative to the treatment side, to allow for treatment of adjacent areas to the treatment area and reduce the problems associated with non-overlapping treatments.

By designing the tip such that heated air is not only delivered along an axis of the tip towards the subject's skin/scalp, but also towards a treatment side (as defined above) perpendicular to the axis of the tip, and towards one or more adjacent regions positioned to one or both sides of the treatment side, elimination or at least reduction of tip placement overlap during treatment may be achieved, thereby enabling a faster treatment pattern and/or higher compliance with use instructions.

Although, as discussed above, it may be preferred to provide tight temperature control of the heated air delivered from the device, paradoxically, in some embodiments, it may be simultaneously preferred to provide a degree of non-uniformity of the air exit temperature at the tines within the tight temperature range. For example, in some embodiments, by setting the target exit temperature to a central location within the range, such as about 57.5 degrees Celsius, a small degree of non-uniformity of tine exit temperature from the device may be allowed while keeping the temperature at all ports in a desired range, such as about 56 degrees Celsius to about 59 degrees Celsius. However, designing the air mixing to deliver air that takes full advantage of a preferred, relatively tight, temperature range, may further require other features/elements disclosed herein, such as the air mixing chamber/zone disclosed herein and/or the preferred tip and/or tine configuration. Combined with slight rotation of the device by the operator, a narrow mix of temperatures may provide hot and cool spots that may promote ectoparasite killing efficacy, patient comfort, and/or compliance.

In some embodiments and implementations, process and/or features may be provided for ensuring, or at least reducing the possibility of, uses of an applicator tip more than a predetermined number of times. For example, in some embodiments, the device may be configured to prohibit or at least inhibit use of a tip more than once, or for more than a predetermined time period.

For example, some embodiments may be configured with one or more microchips or microchip assemblies that may be electrically coupled to a base unit when the tip is coupled with the applicator device. This technology may be one-wire, radio-frequency identification (“RFID”), or any other suitable technology that enables a device control board to recognize the tip, authenticate the tip, and/or read the amount of time and/or number of uses stored on the tip. For example, some embodiments may be configured to actuate a clock or counter upon coupling the tip with the device. Alternatively, or additionally, the device may be configured to actuate a clock upon starting the device, such as upon flipping a switch to turn on a heat element and/or fan within the device. Thus, upon detecting that a threshold number of uses and/or amount of time have been exceeded, the device may be configured to disable the device for use with the current tip, actuate a warning light or other visual or audible indicator, display a message to a user, and/or transmit a message to a supervisor or related entity.

In some embodiments, the device may be configured to display the amount of tip time used and/or remaining to the user through one of many methods, including a row of LED lights, a physical display of a clock or time, or sound indicators. The tip may be programmed at the time of manufacture with one or more authentication codes and enough time to enable treatment of a single patent. As treatment occurs, the tip lifetime counts down until finally it expires. Upon expiration of the threshold, such as treatment time or number of uses, the base unit may be configured to write encrypted code to a microchip on the tip such that it cannot be used again.

In some embodiments, the tip may be programmed such that it can be recognized and read by any device configured to allow for coupling of the tip. For example, if a tip with 50% of its time left is removed from a first device, it will be recognized by the first device, and preferably by any other device with which the tip may be coupled. Thus, a programmable controller on any such device may be configured to read the microchip on the device, recognize the remaining time or other usage figure(s), and display it accordingly.

Some embodiments may further comprise various ergonomic features designed to improve the ability of the device to be used in treatment of ectoparasites without causing undue strain upon the person applying the treatment. For example, some embodiments may comprise a handle and/or one or more grip areas for the operator to use as they move the device through its pattern. Lice tend to concentrate at the back of the head between the ears and near the scalp where there is often humidity produced by the human head. When the device is used on this region it is most comfortably held in a horizontal position. However, the entire head needs to be treated, and the device is typically in a vertical position when treating the top of the scalp. It therefore may be important to give the operator grip points to comfortably enable both the horizontal and vertical device orientations, as well as those that are in between these two orientations. Such grip features preferably also enable the operator's delicate placement of the tip applicator against the head and facilitate device rotation within a treatment spot while also enabling the operator's comfort throughout the procedure.

As such, some preferred embodiments may be equipped with a handle on the underside of the device, as seen in figures discussed in greater detail below. Various different forms for the handle will be apparent to those of ordinary skill in the art after having received the benefit of this disclosure, but preferably the handle is configured to enable operator comfort when the device is in a horizontal use position.

In some embodiments, a handle may also be provided on the top side of the device to provide alternative gripping positions for the user. A top handle may be useful to allow an operator to hold the device comfortably and with finesse while the device is in a vertical position.

Various other grip features may also be included on the device. For example, in some embodiments, various grooves or indents may be provided on the lower handle, upper handle, and/or sides of the device to facilitate gripping when the device is in a position in between vertical and horizontal. In addition, in some embodiments, the upper handle may be configured to allow a user to grip the device around a neck feature while inserting their hand under the upper handle. Preferably, the upper handle is configured so as to enable the operator to release their grip on the device to rest or wiggle the fingers mid-treatment, essentially letting go of the device, without disrupting the treatment as the handle wedges the hand such that the user does not need to grip the device with fingers when the hand is slid under the upper handle.

Some embodiments may further comprise one or more noise attenuation features and/or or components. In some such embodiments, these features/components may be positioned at or near the rear of the device, since this would typically be the area closer to an operator's ears during treatment. Noise can be mitigated with a variety of sound reduction features, such as one or more screens placed behind the blower. In some such embodiments, such screen(s) may comprise hole patterns and/or designs that are not identical and/or feature different hole sizes that are big enough not to impede air flow while physically blocking some of the sound transmission, since it is preferable that the noise attenuation features allow for adequate airflow into the device. In a particular preferred embodiment, the presence of two overlapping screens was found to reduce the noise measured at the back of the device (sound meter held about 12 inches away) by at least about 10 dBA.

The embodiments of the disclosure may be best understood by reference to the drawings, wherein like parts may be designated by like numerals. It will be readily understood that the components of the disclosed embodiments, as generally described and illustrated in the figures herein, could be arranged and designed in a wide variety of different configurations. Thus, the following detailed description of the embodiments of the apparatus and methods of the disclosure is not intended to limit the scope of the disclosure, as claimed, but is merely representative of possible embodiments of the disclosure. In addition, the steps of a method do not necessarily need to be executed in any specific order, or even sequentially, nor need the steps be executed only once, unless otherwise specified. Additional details regarding certain preferred embodiments and implementations will now be described in greater detail with reference to the accompanying drawings.

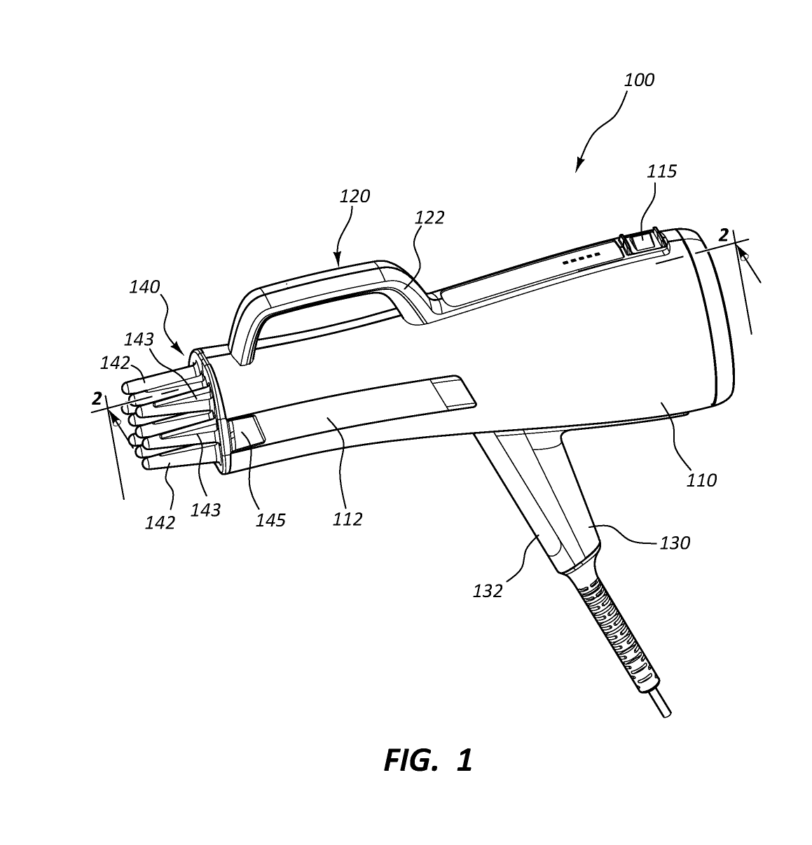

FIG. 1 is a perspective view of an embodiment of an ectoparasite treatment device 100 utilizing heated air to desiccate the ectoparasites and their eggs. Device 100 comprises a handheld ectoparasite treatment device. In other words, device 100 is configured to allow for a typical operator to hold the device with a single hand during a treatment of a subject/patient such that the other hand can be free to manipulate the subject's hair, for example.

Device 100 comprises a housing 110 and two handles, namely an upper handle 120 and a lower handle 130. Upper handle 120 is positioned to extend from an upper surface of housing 110 of device 100. Upper handle 120 may be configured to allow a user to grip the device during certain treatment positions, such as with the device 100 in a vertical position. In some embodiments, the “neck” region of the device below the upper handle 120 may be configured and/or comprise dimensions to allow a user to grasp the device with one hand with their fingers underneath upper handle 120.

Lower handle 130 extends from a lower surface of housing 110 and may be angled towards the rear end of the device 100 such that lower handle 130 does not extend perpendicular from the lower surface of the housing 110, as shown in FIG. 1.

One or more ergonomic gripping features may also be included on the handles and/or housing 110. For example, device 100 comprises indents 112 extending along opposing sides of housing 110 that may facilitate grasping the device 110 with the operator's hand underneath upper handle 120, as described above. In some embodiments, a portion of indents 112 may also allow for receipt of an element from tip 140 to allow for coupling and uncoupling of tip 140 with device 100. For example, in FIG. 1, a coupling member 145 can be seen extending into a portion of indent 112. Coupling member 145 may be coupled with indent 112, or otherwise with device 100, by way of, for example, a friction fit, a snap-fit, tongue-and-groove elements, pins, screws, etc. It should be understood that, although not visible in FIG. 1, tip 140 may comprise another coupling member 145 on the opposite side of housing 110 and, similarly, housing 110 may comprise another indent 112 on the opposite side.

Similarly, gripping pads may be provided on one or both handles, such as gripping pad 122 on upper handle 120 and gripping pad 132 on lower handle 130. As shown in FIG. 1, gripping pad 122 may be positioned on a lower surface of upper handle 120 and gripping pad 132 may be positioned on a distal surface of lower handle 130, since these surfaces would typically receive a majority of the forces associated with gripping the device 100 during operation.

Housing 110 may further comprise a display 115. As described in greater detail below, display 115 may be used to track a usage parameter, such as a current usage time and/or time to expiration for tip 140. Display 115 may also, or alternatively, be configured to display information regarding the expiration of the allotted use time for a particular tip 140, air temperature information, etc.

Tip 140 is positioned on a distal end of housing 110. In preferred embodiments, tip 140 is removable from housing 110 and may be disposable such that new tips may be coupled with housing 110 following treatment, as described in greater detail below.

Tip 140 comprises a plurality of tines 142. At least a subset of the tines 142, and preferably all of the tines 142, comprise ports 143 configured to deliver heated air therethrough for use in treatment of an ectoparasite infestation. The tips of the tines 142 may be plugged or substantially solid. Such a configuration may help to minimize or prevent the potential for burns and/or thermal discomfort, since it may be preferred that tines 142 contact the scalp/skin of a patient during treatment. The tines 142 may therefore be made up of a material having a low thermal conductivity to further avoid burns and/or thermal irritation. The features and functionality of the tip 140 will be described in greater detail below in connection with other figures.

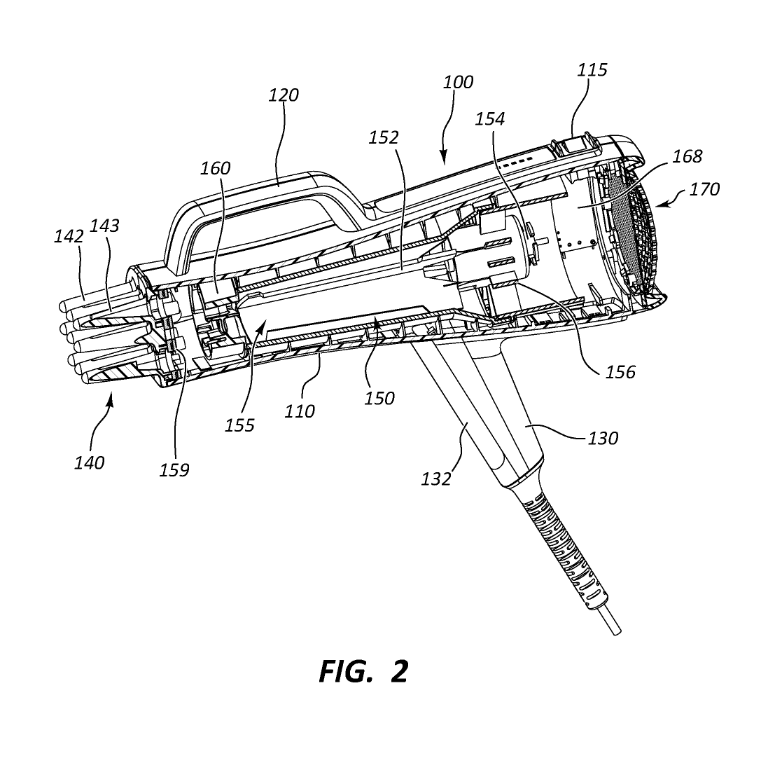

FIG. 2 is a cross-sectional view of device 100 depicting certain internal components of the device. More particularly, FIG. 2 shows a mixing chamber 155 defined by an internal housing 158. A heating assembly 150 is positioned within housing 158. Heating assembly 150 may comprise a heating coil 152, which may comprise a plurality of heating coil blades or other members extending along the length of the mixing chamber 155. A fan 156 may be positioned adjacent to the heating coil 152, such that heated air generated by the heating coil 152 may be directed towards a diffuser 160. As described in greater detail below, diffuser 160 may be configured to receive heated air from fan 156 or another suitable blower and to mix the heated air to increase uniformity of temperature of the heated air delivered from tip 140. A motor 154 may be provided to power fan 156.

One or more noise attenuation features and/or components may be provided, preferably at the proximal end of treatment device 100. For example, device 100 comprises a noise attenuation assembly 170, which may comprise multiple screens having apertures of different shapes and sizes or apertures that otherwise do not precisely line up with one another such that air can travel therethrough but the noise from the internal components of the device 100 may be reduced.

Device 100 may further comprise one or more temperature sensors 159 configured to sense a temperature of the heated air. Preferably, temperature sensor(s) 159 is positioned at the distal end of device 100 adjacent to tip 140 such that the air being delivered through ports 143 may be precisely controlled. In some embodiments, the temperature sensor(s) 159 is positioned so as to extend or otherwise be positioned between and/or adjacent to most, or all, of the ports 143. In certain preferred embodiments, the temperature sensor(s) 159 is positioned such that heated air exiting diffuser 160 directly impacts the temperature sensor(s) 159 without contacting any intervening objects. This may allow for further improvement of the ability of the device 100 to tightly control the temperature of the heated air being delivered through tip 140. In some embodiments, this feature, in combination with one or more of the other elements disclosed herein, may allow for precision of the temperature of the air delivered through tip 140 to vary by no more than about 2 degrees Celsius in either direction. Thus, some embodiments may be configured such that the heated air delivered through tip 140 is about 57.5 degrees Celsius+/−about 2 degrees Celsius, as measured at any of the ports 143 on tip 140.

Temperature control electronics 168, may also be provided. Temperature control electronics 168 may comprise, for example, a printed circuit board (“PCB”) or other suitable hardware, firmware, software, or any combination thereof. Temperature control electronics 168 is operatively coupled with temperature sensor(s) 159 and heating assembly 150. Thus, temperature control electronics 168 may be configured to receive temperature data from the temperature sensor(s) 159 and adjust the heating assembly 150 such that the heated air delivered through tip 140 is maintained between a minimum temperature of about 54 degrees Celsius and a maximum temperature of about 62 degrees Celsius, as measured at each of the ports 143. In some embodiments, temperature control electronics 168 may be configured to receive temperature data from the temperature sensor(s) 159 and adjust the heating assembly 150 such that the heated air delivered through tip 140 is maintained between a minimum temperature of about 56 degrees Celsius and a maximum temperature of about 59 degrees Celsius, as measured at each of the ports 143. It has been discovered that the temperature ranges disclosed herein may provide the best combination of efficacy (i.e., ability to kill lice and lice eggs) and safety/comfort. In some embodiments, temperature control electronics 168 may also, or alternatively, be used to facilitate electronic communication with tip 140, so as to control use of tip 140 as desired, such as to prevent multiple uses of tip 140 or prevent tip 140 from being used for longer than a predetermined usage time, for example. These features of certain preferred embodiments will be discussed in greater detail below. In alternative embodiments, separate electronic components may be used to control usage parameters of tip 140.

In some preferred embodiments, the temperature sensor(s) 159 is configured with an accuracy of no more than about +/−0.1° C. Similarly, in preferred embodiments and implementations, temperature adjustment frequency by temperature control electronics 168 and/or heating assembly 150, for example, may be at least once every 1 second. More preferably, temperature adjustment frequency may be at least once every 0.1 seconds. Even more preferably, temperature adjustment frequency may be at least once every 1/60th of a second (60 Hz). Some embodiments may incorporate a proportional-integral-derivative (“PID”) algorithm. For example, some embodiments may comprise a PID controller or another control loop feedback mechanism.

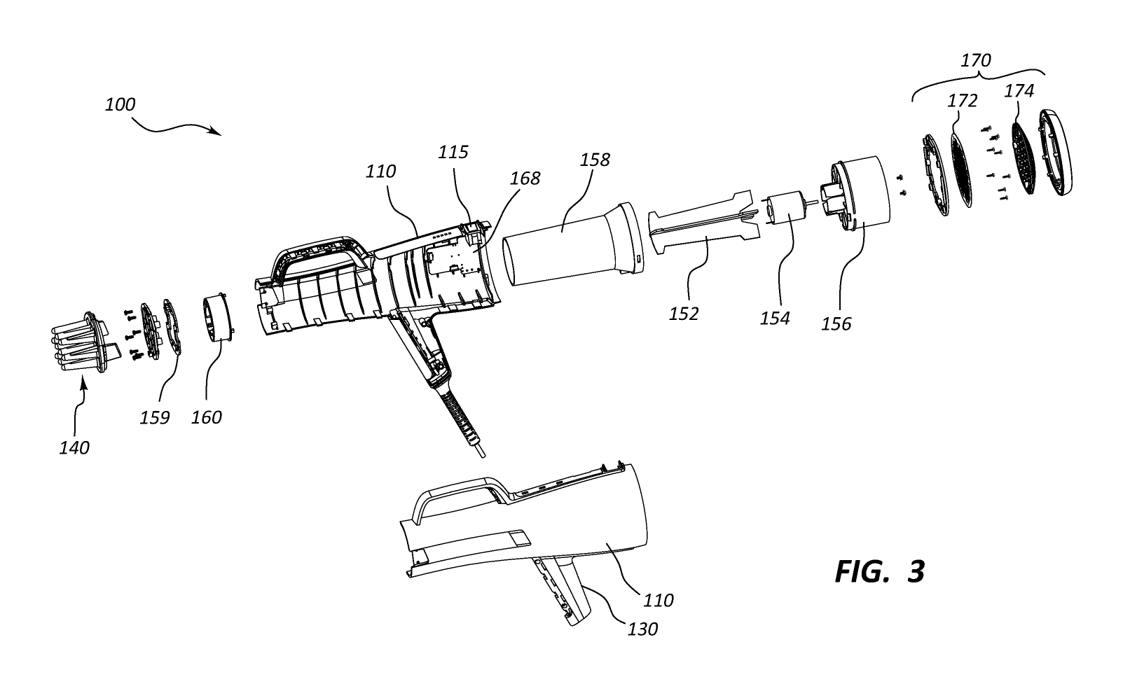

FIG. 3 is an exploded view of device 100, which allows for better illustration of certain internal components of the device. For example, as best seen in FIG. 3, noise attenuation assembly 170 may comprise a first screen 172 comprising a first plurality of openings and a second screen 174 positioned adjacent to the first screen 172 and having a second plurality of openings. As previously mentioned the openings of the two screens are preferably misaligned, sized differently, and/or shaped differently to allow for reduction of the noise caused by the device 100. The present inventors have discovered that, in some preferred embodiments, the presence of two overlapping screens as described herein can reduce the noise measured at the back of the device (sound meter held about 12 inches away from proximal end) by at least about 10 dBA.

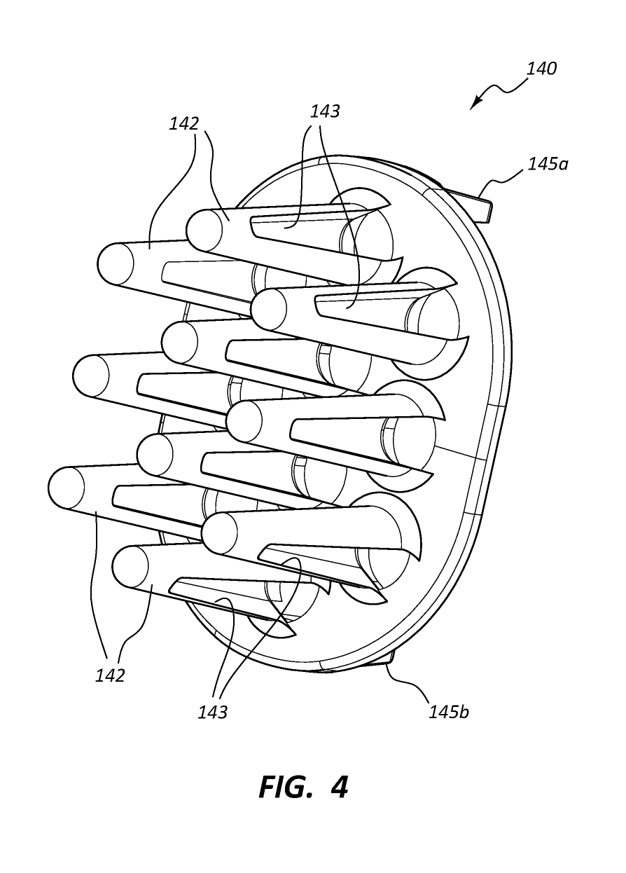

FIG. 4 is a perspective view of a tip 140 according to some embodiments. As previously mentioned, preferably tip 140 is configured to be removed from device 100, and may be disposable to allow for a new tip to be used for each treatment, or to allow for replacement of tips following a desired number of treatments and/or treatment time. Thus, tip 140 comprises opposing coupling members 145a and 145b. Coupling members 145a/145b in the depicted embodiment comprise opposing tabs, which may be configured to be received in opposing detents or recesses formed within housing 110 to couple tip 140 with the rest of device 100. Coupling members 145a/145b may be configured to flex so as to allow for a snap-fit or friction-fit coupling between housing 110 and tip 140. Of course, it should be understood that tabs or other coupling members may instead be on the housing, and the detents/recesses on the tip 140, if desired. It should also be understood that a variety of alternative means for coupling a removable tip with an ectoparasite treatment device may be provided, as those having ordinary skill in the art will appreciate after having received the benefit of this disclosure.

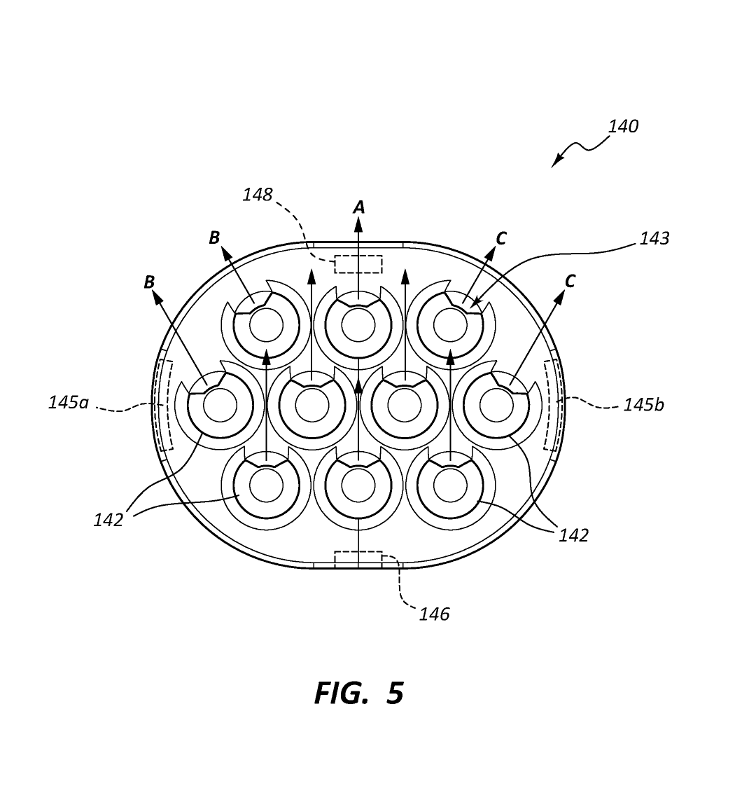

FIG. 5 is a top plan view of tip 140 and also includes arrows to indicate the directionality of heated air through ports 143 of tines 142. As illustrated in this figure, tip 140 may comprise different sets of tines 142 configured to deliver heated air in different directions to optimize treatment and allow for avoiding extensive overlap of treatment patterns on a subject/patient. More particularly, a first subset of the tines 142 comprise ports 143 that are configured to direct heated air in a first, distal or forward direction from the perspective of the top plan view of FIG. 5. The heated air from this first subset of tines 142 is indicated by arrows “A” in FIG. 5.

A second subset of ports 143 are each configured to direct heated air in a second direction extending at an angle relative to the first direction. The heated air from this second subset of tines 142 is indicated by arrows “B” in FIG. 5 and extends at an angle to the left relative to the first direction from the perspective of FIG. 5. In some embodiments, the angle with which the heated air from the second subset of ports 143 (direction B) extends relative to direction A is between about 30 and about 90 degrees. In more preferred embodiments, the angle with which the heated air from the second subset of ports 143 (direction B) extends relative to direction A is about 45 degrees. Since it is expected that air delivered from each of the various ports will spread out, these angles should be measured from the center of each respective port.

A third subset of ports 143 are each configured to direct heated air in a third direction extending at a second angle relative to the first direction and in an opposite direction relative to the first angle. The heated air from this third subset of tines 142 is indicated by arrows “C” in FIG. 5 and extends at an angle to the right relative to the first direction from the perspective of FIG. 5. In some embodiments, the angle with which the heated air from the third subset of ports 143 (direction C) extends relative to direction A is between about 30 and about 90 degrees. In more preferred embodiments, the angle with which the heated air from the third subset of ports 143 (direction C) extends relative to direction A is about 45 degrees.

In some preferred embodiments, tip 140 may be configured such that no heated air, or at least substantially no heated air, is directed opposite from the heated air extending in direction A. This may be useful for a number of reasons, such as for avoiding having heated air directed towards an operator during treatment, for example.

As also shown in FIG. 5, tip 140 may further comprise an alignment notch 146, which may be configured to engage a corresponding groove formed on housing 110 or otherwise on device 100. This may ensure that tip 140 is properly coupled with device 100 in the correct rotational position such that the heated air delivery pattern described above is properly applied to a patient/subject during treatment. Notch 146 may also be used, along with coupling members 145a/145b, to further secure tip 140 in place on housing 110.

Tip 140 may further comprise a memory element, such as microchip 148. Microchip 148 may be configured to receive and store tip usage parameter data, such as the time elapsed during which tip 140 has been used during treatment, the number of treatment sessions, etc. Microchip 148 may therefore be used to prevent, or at least reduce the possibility, that tip 140 will be used more than a predetermined number of times or for more than a predetermined treatment time. This may be done, for example, by providing for communication, preferably wireless communication, between microchip 148 and an electrical component located on the base and/or housing portion of device 100, such as electronics/PCB 168.

In some embodiments, PCB 168 may comprise a wireless communication transmitter or transceiver, such as, for example, Near Field Communication (“NFC”) standards, IEEE's 802.11 standards, Bluetooth®, ultra-wide band (“UWB”), Zigbee®, RFID, and/or any other suitable wireless communication protocols or combinations thereof, or any other technology that enables a device to communicate with a tip. In some embodiments, PCB 168 may be configured to query tip 140 to obtain information from tip 140 to recognize the tip 140, authenticate the tip 140, and/or read the tip usage parameter data stored on the tip 140. Some embodiments may further be configured to actuate a clock or counter upon coupling the tip 140 and detecting the tip 140 by the device 100. Alternatively, or additionally, the device 100 may be configured to actuate a clock upon starting the device 100, such as upon flipping a switch to turn on a heat element and/or fan within the device 100. Thus, upon detecting that a threshold number of uses and/or amount of time have been exceeded, PCB 168 may be configured to disable the device for use with the current tip 140, actuate a warning light or other visual or audible indicator, display a message to a user, such as on display 115, and/or transmit a message to a remote computing device.

Microchip 148 of tip 140 may be programmed with one or more authentication codes or other codes, which codes may record information that may be accessed by PCB 168, such as a predetermined treatment time and/or number of allowed treatments. As treatment occurs, the tip lifetime may count down until finally it expires. Upon expiration of the threshold, such as treatment time or number of uses, PCB 168 may be configured to write encrypted code to microchip 148 on tip 140 such that it cannot be used again and/or such that attempts to use the same tip 140 may result in another suitable action, actuation of a warning light or other visual or audible indicator, display of a message to a user, and/or transmission of a message to a remote computing device.

In some embodiments, microchip 148 may be programmed such that it can be recognized and read by any other device configured to allow for coupling of a similar line of tips. Thus, in some such embodiments, upon being removed from a first device and coupled with a second device, PCB 168 or another programmable controller on the second device may be configured to read microchip 148, recognize any remaining time or other usage data, and act on the received usage data, such as displaying the remaining treatment time available, activating or deactivating the device, etc.

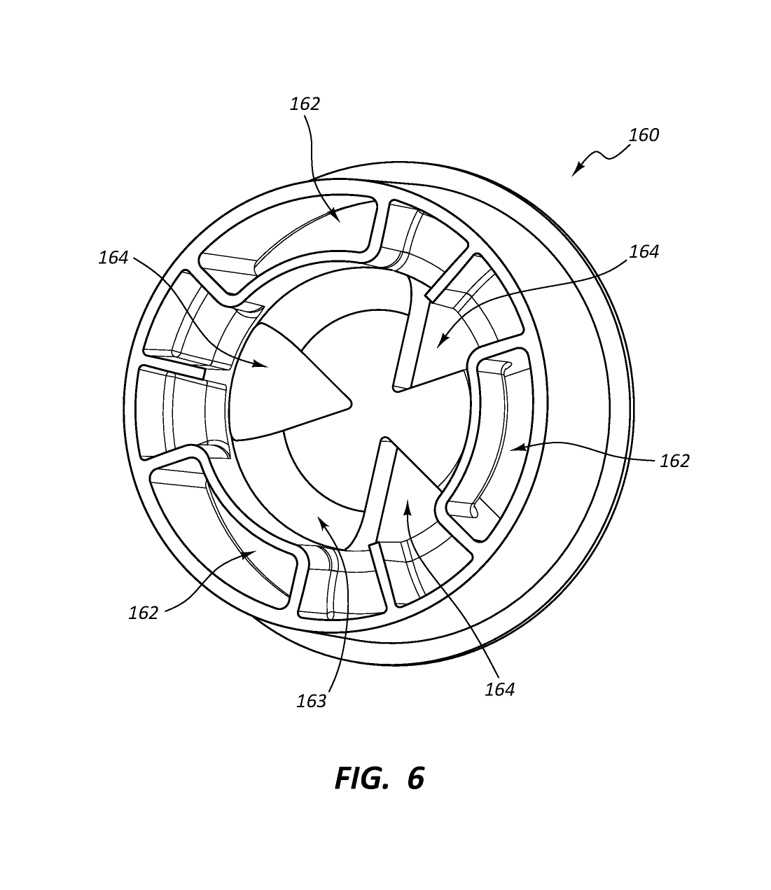

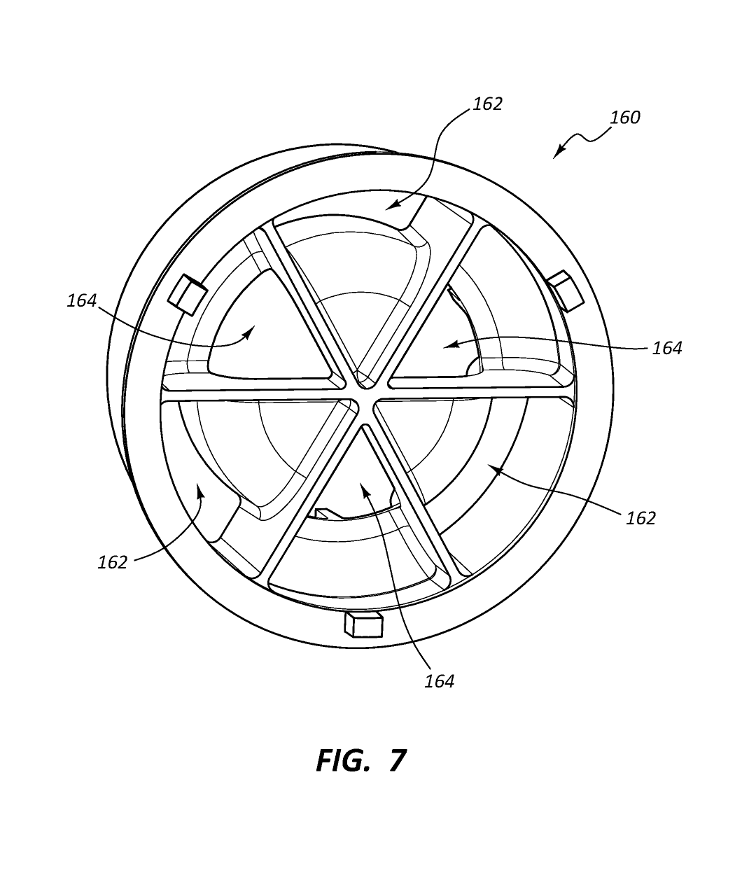

FIGS. 6 and 7 are perspective views of opposing sides of air diffuser 160. FIG. 6 depicts diffuser 160 from a proximal side and FIG. 7 from a distal side. Diffuser 160 may be positioned within housing 110 and be configured to receive heated air from a fan or other blower, such as fan 156, and deliver the heated air into tip 140. Preferably, diffuser 160 is configured to mix the heated air being delivered therethrough to increase uniformity of temperature of the heated air being delivered through ports 143 of tines 142.

As shown in FIGS. 6 and 7, diffuser 160 may comprise two sets of air flow apertures. More particularly, a first set of apertures 164 may be configured to receive heated air therethough from fan 156 and/or mixing chamber 155, and a second set of apertures 162 may be configured to deliver heated air to tip 140. As also shown in these figures, the first set of apertures 162 is misaligned with respect to the second set of apertures 164. More particularly, apertures 162 are positioned about a peripheral edge of diffuser 160 and apertures 164 are positioned radially inward of apertures 162. In addition, it can be seen that apertures 162 are shaped differently from apertures 164. Finally, in some embodiments, a second mixing chamber 163 may be defined by diffuser 160, such that mixing chamber 163 is fluidly coupled with both sets of apertures 162/164 and such that air entering mixing chamber 163 from apertures 164 is forced to be redirected into mixing chamber 163 prior to exiting apertures 162.

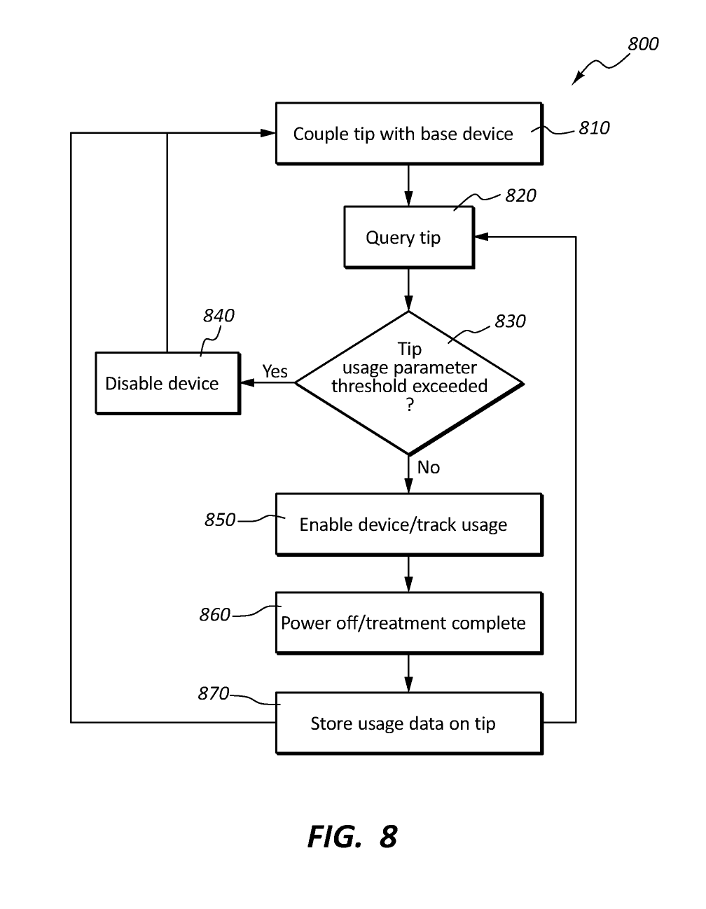

FIG. 8 is a flow chart illustrating an example of a method 800 for treating an ectoparasite infestation according to some implementations of the invention. Step 810 in method 800 comprises coupling a removable tip with a housing/base of an ectoparasite treatment device configured to deliver heated air to desiccate ectoparasites and ectoparasite eggs. As described above, the ectoparasite treatment device may comprise a housing and the removable tip may comprise a plurality of tines, wherein at least a subset of the tines (in some implementations, all) comprises ports configured to deliver heated air therethrough.

After coupling the tip with the housing/base, the tip may be queried at step 820, such as by interrogation of an RFID tag positioned in the tip, sending another wireless signal to the tip, such as to a microchip and/or memory component located on the tip, as previously described. At 830, a determination may be made as to whether a tip usage parameter threshold has been exceeded. For example, in some implementations, the device may communicate with a component on the tip to determine whether the tip has been used before and, if so, whether an allotted treatment time and/or number of allowed uses/treatments has been exceeded. More particularly, in some implementations, step 830 may comprise transmitting a treatment time or other tip usage parameter data from the tip, such as from a microchip or other memory element on the tip; comparing the treatment time or other tip usage parameter data with a threshold; and upon determining that the treatment time or other tip usage parameter data has not exceeded the threshold, allowing the ectoparasite treatment device to operate.

If the tip usage parameter threshold has been exceeded, method 800 proceeds to step 840, at which point the device may be disabled. In alternative implementations, step 840 may comprise one or more of storing an encrypted code on the tip, such as on a memory element located on the tip indicating that usage time for the tip has expired, disabling the device for use with the queried tip, generating an audible notification, generating a visible notification, and transmitting a wireless signal indicating that the tip usage parameter threshold has been exceeded. A new tip may then be coupled with the device at step 810.

If, at 830, it is determined that the tip usage parameter threshold has not been exceeded, method 800 proceeds to step 850, at which point the device may be enabled for treatment and/or a tip usage parameter may be initiated for tracking. For example, in some implementations, step 850 may comprise tracking a usage time of the ectoparasite treatment device during a treatment session. In some implementations, step 850 may additionally, or alternatively, comprise tracking a power cycle or other indication of initiation of a treatment session.

Following the treatment, the device may be powered off at step 860. Upon detecting a power cycle, in some implementations, method 800 may then proceed to step 870 at which point the usage time/data may be stored on a component of the tip, such as on a microchip and/or memory element positioned on the tip. In alternative implementations, data may be stored on the tip at one or more points during the treatment itself.

Following step 870, the tip may be removed from the device to allow another tip to be coupled with the base/housing at step 810. Alternatively, after storing usage data on the tip, the tip may be queried again at step 820 to ensure that a tip usage parameter threshold is not exceeded during treatment. Thus, those of ordinary skill in the art will appreciate that step 870 may be performed prior to step 860 and/or steps 820 and 830 may be performed multiple times during a treatment session.

The above description fully discloses the invention including preferred embodiments thereof. Without further elaboration, it is believed that one skilled in the art can use the preceding description to utilize the invention to its fullest extent. Therefore the examples and embodiments disclosed herein are to be construed as merely illustrative and not a limitation of the scope of the present invention in any way.

It will be obvious to those having skill in the art that many changes may be made to the details of the above-described embodiments without departing from the underlying principles of the invention. The scope of the present invention should, therefore, be determined only by the following claims.

1. A handheld ectoparasite treatment device, comprising:
a handle configured to allow an operator to grip the handle and operate the handheld ectoparasite treatment device with a single hand;
a housing coupled with the handle;
a heating assembly positioned within the housing, wherein the heating assembly is configured to generate heated air for treatment of ectoparasites;
a blower;
an air diffuser positioned within the housing and configured to receive heated air from the blower, wherein the air diffuser is configured to mix the heated air to increase uniformity of temperature of the heated air;
a tip coupleable with the housing, wherein the tip comprises a plurality of tines, wherein at least a subset of the tines comprises ports configured to deliver heated air therethrough, wherein the tip is fluidly coupled with the air diffuser, and wherein the tip is configured to be removable from the handheld ectoparasite treatment device;
a temperature sensor configured to sense a temperature of the heated air; and
temperature control electronics operatively coupled with the temperature sensor and the heating assembly, wherein the temperature control electronics is configured to receive temperature data from the temperature sensor and adjust the heating assembly such that the heated air delivered through the tip is maintained between a minimum temperature of about 54 degrees Celsius and a maximum temperature of about 62 degrees Celsius, as measured at each of the ports.

2. The handheld ectoparasite treatment device of claim 1, wherein the temperature control electronics is configured to receive temperature data from the temperature sensor and adjust the heating assembly such that the heated air delivered through the tip is maintained between a minimum temperature of about 56 degrees Celsius and a maximum temperature of about 59 degrees Celsius, as measured at each of the ports.

3. The handheld ectoparasite treatment device of claim 1, wherein the air diffuser comprises:
a first set of apertures configured to receive heated air therethough;
a mixing chamber fluidly coupled with the first set of apertures; and
a second set of apertures configured to deliver heated air from the mixing chamber out of the air diffuser, wherein the first set of apertures is misaligned with respect to the second set of apertures.

4. The handheld ectoparasite treatment device of claim 1, further comprising:
a memory element positioned on the tip, wherein the memory element is configured to receive and store tip usage parameter data; and
a controller configured to be communicatively coupled with the memory element when the tip is coupled with the housing, wherein the controller is configured to receive tip usage parameter data and alter at least one function of the handheld ectoparasite treatment device in accordance with the tip usage parameter data.

5. The handheld ectoparasite treatment device of claim 4, wherein the tip usage parameter data comprises tip usage time, and wherein the controller is configured to receive the tip usage time from the memory element and, upon determining that the tip usage time has exceeded a threshold treatment time, alter the at least one function of the handheld ectoparasite treatment device.

6. The handheld ectoparasite treatment device of claim 5, wherein the controller is configured to alter the at least one function of the handheld ectoparasite treatment device by at least one of:
storing an encrypted code on the memory element indicating that the tip usage time has expired;
disabling the ectoparasite treatment device for use with the tip;
generating an audible notification;
generating a visible notification; and
transmitting a wireless signal indicating that the threshold treatment time has been exceeded.

7. An ectoparasite treatment device, comprising:
a heating assembly configured to generate heated air for treatment of ectoparasites;
a blower;
an air diffuser configured to receive heated air from the blower, wherein the air diffuser is configured to mix the heated air to increase uniformity of temperature of the heated air;
a tip comprising a plurality of elongated tines, wherein at least a subset of the tines comprises ports configured to deliver heated air therethrough, wherein a first subset of the ports are each configured to direct heated air in a first direction relative to a cross-section of the tip extending perpendicular to an axis of the tines, wherein a second subset of the ports are each configured to direct heated air in a second direction extending at a first angle relative to the first direction, wherein a third subset of the ports are each configured to direct heated air in a third direction extending at a second angle relative to the first direction and in an opposite direction relative to the first angle, and wherein the tip is fluidly coupled with the air diffuser.

8. The ectoparasite treatment device of claim 7, further comprising:
a temperature sensor configured to sense a temperature of the heated air; and
temperature control electronics operatively coupled with the temperature sensor and the heating assembly, wherein the temperature control electronics is configured to receive temperature data from the temperature sensor and adjust the heating assembly such that the heated air delivered through the tip is maintained between a minimum temperature of about 54 degrees Celsius and a maximum temperature of about 62 degrees Celsius, as measured at each of the ports.

9. The ectoparasite treatment device of claim 8, wherein the temperature control electronics is configured to receive temperature data from the temperature sensor and adjust the heating assembly such that the heated air delivered through the tip is maintained between a minimum temperature of about 56 degrees Celsius and a maximum temperature of about 59 degrees Celsius, as measured at each of the ports.

10. The ectoparasite treatment device of claim 7, wherein the tip is configured to be removable from the handheld ectoparasite treatment device, and wherein the ectoparasite treatment device is configured to allow for being coupled with a different tip.

11. The ectoparasite treatment device of claim 7, wherein the first angle is about 45 degrees.

12. The ectoparasite treatment device of claim 7, wherein the each of the ports is configured such that at least substantially no heated air is directed in a direction opposite from the first direction.

13. The ectoparasite treatment device of claim 7, wherein the air diffuser comprises:
a first set of apertures configured to receive heated air therethough;
a mixing chamber fluidly coupled with the first set of apertures; and
a second set of apertures configured to deliver heated air from the mixing chamber out of the air diffuser, wherein the first set of apertures is misaligned with respect to the second set of apertures.

14. A method for treating an ectoparasite infestation, the method comprising the steps of:
obtaining an ectoparasite treatment device configured to deliver heated air to desiccate ectoparasites and ectoparasite eggs, wherein the ectoparasite treatment device comprises:
a housing; and
a tip comprising a plurality of tines, wherein at least a subset of the tines comprises ports configured to deliver heated air therethrough;
coupling the tip with the housing;
validating a tip usage parameter associated with the tip; and
using the ectoparasite treatment device to apply heated air to the hair and scalp of a human patient during a treatment session, wherein the heated air is maintained between a minimum temperature of about 54 degrees Celsius and a maximum temperature of about 62 degrees Celsius during the treatment session.

15. The method of claim 14, wherein the tip usage parameter comprises a treatment time.

16. The method of claim 15, wherein the step of validating the tip usage parameter comprises:
transmitting the treatment time from a memory element on the tip;
comparing the treatment time with a threshold treatment time; and
upon determining that the treatment time has not exceeded the threshold treatment time, allowing the ectoparasite treatment device to operate.

17. The method of claim 16, further comprising, upon determining that the treatment time has exceeded the threshold treatment time, performing at least one of the following actions:
storing an encrypted code on the memory element indicating that usage time for the tip has expired;
disabling the ectoparasite treatment device for use with the tip;
generating an audible notification;
generating a visible notification; and
transmitting a wireless signal indicating that the threshold treatment time has been exceeded.

18. The method of claim 14, further comprising:
tracking a usage time of the ectoparasite treatment device during the treatment session; and
storing the usage time on a component of the tip.

19. The method of claim 18, further comprising:
removing the tip from the housing;
coupling a tip with a second ectoparasite treatment device; and
transmitting the usage time from a memory element on the tip to a controller of the second ectoparasite treatment device.

20. The method of claim 19, further comprising:
comparing the usage time with a threshold treatment time; and
upon determining that the usage time has not exceeded the threshold treatment time, allowing the second ectoparasite treatment device to operate.
